(19) United States
(12) Patent Application Publication
(10) Pub. No.: US 2026/0285823 A1
(43) Pub. Date: Sep. 24, 2026

(54) METHOD FOR SYNTHESISING 5-HYDROXYMETHYLFURFURAL

(71) Applicant: IFP ENERGIES NOUVELLES, RUEIL-MALMAISON (FR)

(72) Inventors: Kim LARMIER, RUEIL-MALMAISON CEDEX (FR); Gerhard PIRNGRUBER, RUEIL-MALMAISON CEDEX (FR); Yuna HAN, RUEIL-MALMAISON CEDEX (FR)

(73) Assignee: IFP ENERGIES NOUVELLES, RUEIL-MALMAISON (FR)

(21) Appl. No.: 18/880,383

(22) PCT Filed: Jun. 27, 2023
(86) PCT No.: PCT/EP2023/067408

§ 371 Date: Dec. 31, 2024

(30) Foreign Application Priority Data
Jul. 1, 2022 (EP) 22182679.5

(51) Int. Cl. C07D 307/48; B01J 29/08

(52) U.S. Cl. CPC C07D 307/48; B01J 29/084

(57) ABSTRACT

The present invention relates to a process for dehydrating a hexose-containing feedstock, said process comprising the steps of:
preparing a catalyst by contacting a zeolite containing a series of channels in which the opening is greater than or equal to 10 oxygen atoms (10 MR) and having an Si/Al ratio of at least 10 with a solution comprising an organic compound of formula:

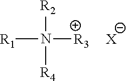

R₁, R₂, R₃, and R₄ being independently selected from linear, branched or cyclic alkyl groups of 1 to 10 carbon atoms, and X⁻ being the hydroxide ion OH⁻;
contacting said dehydration catalyst with said hexose-containing feedstock in at least one solvent, said contacting being carried out at a temperature of between 3° and 160° C. and at a pressure of between 0.0001 and 8.0 MPa.

### TECHNICAL FIELD

The invention relates to a process for producing 5-hydroxymethylfurfural (5-HMF) from a feedstock containing sugars, in particular at least one hexose and preferably fructose, in the presence of at least one solvent, preferably a polar aprotic solvent, and at least one dehydration catalyst comprising a zeolite modified by base treatment in the presence of organic compounds of the tetraalkylammonium hydroxide family.

### PRIOR ART

5-Hydroxymethylfurfural (5-HMF) is a biomass-derived compound that can be upgraded in many ways, such as in specialty chemistry, polymers, or agrochemistry.

The production of 5-HMF by dehydration of hexoses is well known to those skilled in the art and is the subject of intense research. It is known that the use of a Brønsted acid catalyst accelerates the reaction. Catalysts of the zeolite class are especially capable of catalyzing the reaction efficiently.

However, the diffusion of hexoses into the pore structure of zeolites can prove limiting for the kinetics of the reaction, as is described in the publication by Dapsens et al. ((2014) “Hierarchical Sn-MFI zeolites prepared by facile top-down methods for sugar isomerisation” in Catalysis Science & Technology, vol. 4, No. 8, p. 2302). Moreover, according to the publication by Ordomsky et al. ((2012) “The effect of solvent addition on fructose dehydration to 5-hydroxymethylfurfural in biphasic system over zeolites” in Journal of Catalysis, vol. 287, p. 68-75), prolonged contact of sugars with the external surface of zeolites leads to the formation of undesirable polymeric by-products referred to generically as humins that lead to both losses in yield and catalyst inactivation.

It is therefore advantageous to mitigate the limitations on the diffusion of hexoses into zeolites.

The publication by Rac et al. ((2014) “Hierarchical ZSM-5, Beta and USY zeolites. Acidity assessment by gas and aqueous phase calorimetry and catalytic activity in fructose dehydration reaction” in Microporous and Mesoporous Materials, vol. 194, p. 126-134) discloses the use of zeolites modified by base treatment in the presence of sodium hydroxide (NaOH) and tetrapropylammonium bromide in a process for the dehydration of fructose to 5-HMF.

The drawback of the processes of the prior art is the use of NaOH in conjunction with tetraalkylammonium compounds during zeolite modification treatments. This is because the use of NaOH (or other alkali or alkaline earth metal hydroxides) neutralizes the acidity of the zeolite and necessitates additional steps of retro-exchange of Na⁺ (or other alkali or alkaline earth metal salts) after the base treatment, these steps making the process costly.

The present invention thus aims to improve the performance of zeolite catalysts for the production of 5-HMF from sugars, while limiting costs. Specifically, the applicant has discovered that it is possible to use, as catalysts for converting sugars into 5-HMF, zeolites modified by treatment in a basic medium in the presence of at least one organic compound acting as a structure-directing agent of the tetraalkylammonium hydroxide class, without the use of an additional basic compound such as sodium hydroxide, in order to improve catalyst performance in the production of 5-HMF from hexoses. Advantageously, selectivity is improved and the formation of oligomeric by-products is limited, and the process is more economical.

### SUMMARY OF THE INVENTION

The present invention relates to a process for dehydrating a hexose-containing feedstock, said process comprising the steps of:
preparing a catalyst by contacting a zeolite containing a series of channels in which the opening is greater than or equal to 10 oxygen atoms (10 MR) and having an Si/Al ratio of at least 10 with a solution comprising an organic compound of formula:

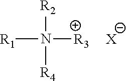

R₁, R₂, R₃, and R₄ being independently selected from linear, branched or cyclic alkyl groups of 1 to 10 carbon atoms, and X⁻ being the hydroxide ion OH⁻;
contacting said dehydration catalyst with said hexose-containing feedstock in at least one solvent, said contacting being carried out at a temperature of between 3° and 160° C. and at a pressure of between 0.0001 and 8.0 MPa.

### DETAILED DESCRIPTION OF THE INVENTION

According to the present invention, the expressions “of between . . . and . . . ” and “between . . . and . . . ” are equivalent and mean that the limiting values of the interval are included in the described range of values. If this is not the case and the limit values are not included in the described range, such a clarification will be provided by the present invention.

For the purposes of the present invention, the various parameter ranges for a given step, such as pressure ranges and temperature ranges, may be used alone or in combination. For example, for the purposes of the present invention, a preferred pressure value range may be combined with a more preferred temperature value range.

Particular embodiments of the invention may be described hereinbelow. They may be employed separately or in combination with one another, with no limitation on combinations where these are technically feasible.

### Feedstock

The feedstock employed in the process according to the invention contains a hexose.

A hexose-containing feedstock is understood as meaning that the hexose may be in monomeric form (monosaccharide) or else be a unit forming part of a disaccharide, oligosaccharide or polysaccharide. A saccharide is a compound also referred to as a sugar.

The hexose is preferably fructose or a fructoside unit.

In one embodiment, the feedstock comprises free fructose, alone or in a mixture with any saccharide species, or comprises any oligosaccharide or polysaccharide feedstock containing one or more fructoside units capable of releasing fructose through one or more hydrolysis steps, possibly in a mixture with other saccharide species. The feedstock treated in the process is preferably crystalline fructose, a syrup containing fructose and glucose, or crystalline sucrose or a sucrose syrup.

The fructose-containing feedstock advantageously comprises fructose in monomeric, oligomeric or polymeric form.

A feedstock containing free fructose in a mixture with any saccharide species means, for example, syrups of the high-fructose corn syrup type containing fructose and glucose in varying proportions (glucose/fructose in ratios by weight or by moles of 58/42, 45/55, 10/90, for example).

A syrup is understood as meaning a solution of sugars, in particular of saccharides, in water having a concentration of at least 30% by weight, preferably at least 50% by weight, preferably at least 70% by weight.

The feedstock may comprise a saccharide comprising one or more fructoside units and one or more non-fructoside units from which fructose may be released through one or more hydrolysis steps, for example oligosaccharides and polysaccharides in which at least one monosaccharide unit is fructose, for example feedstocks such as sucrose, kestose, fructans, oligofructans, and inulin.

The saccharide feedstocks are advantageously capable of releasing monomeric fructose through glycoside hydrolysis, wherein said fructose produced can be converted into 5-HMF.

The oligosaccharide preferably has the empirical formula: (C₆ₘH₁₀ₘ₊₂O₅ₘ₊₁) (C₅ₙH₈ₙ₊₂O₄ₙ₊₁), where m and n are integers, the sum of which is between 2 and 6. The monosaccharide units making up said oligosaccharide are identical or different and at least one unit of formula (C₆ₘH₁₀ₘ₊₂O₅ₘ₊₁) is fructose. By extension, the polysaccharide preferably has the empirical formula (C₆ₘH₁₀ₘ₊₂O₅ₘ₊₁) (C₅ₙH₈ₙ₊₂O₄ₙ₊₁), where m and n are integers, the sum of which is greater than or equal to 7.

### Preparation of the Dehydration Catalyst

The dehydration catalyst is prepared from a zeolite that contains a series of channels in which the opening is greater than or equal to 10 oxygen atoms (10 MR) and having an Si/Al molar ratio of at least 10.

Advantageously, this starting zeolite is referred to as the parent zeolite.

In one embodiment, the opening of the series of channels of the zeolite is equal to 12 oxygen atoms (12 MR).

Preferably, it is a zeolite of the FAU or EMT structural type. Very preferably, it is a dealuminated faujasite, referred to as USY (ultra-stable Y), advantageously having a lattice parameter of less than 24.5 Å.

In a preferred embodiment, the Si/Al molar ratio of the parent zeolite is between 10 and 50, preferably between 12 and 35.

If the Si/Al molar ratio of the parent zeolite is less than 10, a dealumination treatment can be applied, preferably prior to any step of contacting with an organic compound of the tetraalkylammonium hydroxide type, in order to obtain a zeolite having an Si/Al molar ratio in the desired range. This dealumination treatment consists of contacting the zeolite with an acid solution or a solution comprising a complexing agent capable of forming a stable complex with Al³⁺ in solution. These methods are well known to those skilled in the art. Some examples of suitable acids are: HNO₃, HCl, H₂SO₄, CH₃COOH, HOOC—COOH. Examples of complexing agents include EDTA (ethylenediaminetetraacetic acid) in its acid or Na salt form, NTA (nitrilotriacetic acid), etc. The concentration of the acid or complexing agent in the acid solution or complexing agent solution is preferably between 0.1 and 6 mol/l. The volume of solution used is preferably between 3 and 50 ml per gram of zeolite (dry mass of zeolite). The temperature in the dealumination treatment is preferably between 2° and 100° C. The duration of the dealumination treatment is preferably between 15 min and 10 h. After the dealumination treatment, the zeolite is advantageously separated by filtration or centrifugation, dried, preferably at a temperature of between 8° and 150° C., and finally calcined at a temperature of between 450° C. and 600° C.

The dry mass of a zeolite is understood as meaning the mass of said zeolite after treatment at 1000° C.

To improve catalyst performance in the conversion of sugars, preferably fructose, to 5-HMF, the zeolite (optionally after adjusting the Si/Al ratio by dealumination) is contacted with a solution comprising an organic compound of formula:

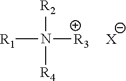

R₁, R₂, R₃, and R₄ being independently selected from linear, branched or cyclic alkyl groups of 1 to 10 carbon atoms, and X⁻ being the hydroxide ion OH⁻.

Said organic compound is more specifically referred to as a tetraalkylammonium hydroxide.

The groups R₁, R₂, R₃, and R₄ may be identical or different.

In one particular embodiment, said organic compound contains at least one group R₁, R₂, R₃ or R₄ selected from methyl, ethyl, propyl and butyl groups. In a preferred embodiment, the organic compound is selected from tetramethylammonium hydroxide, tetraethylammonium hydroxide, tetrapropylammonium hydroxide, and tetrabutylammonium hydroxide.

In a preferred embodiment, said organic compound contains at least one group R₁, R₂, R₃ or R₄ selected from propyl and butyl groups.

In a very preferred embodiment, the organic compound is selected from tetrapropylammonium hydroxide and tetrabutylammonium hydroxide.

This treatment of the zeolite, by contacting with a tetraalkylammonium, makes it possible to modify the acidity of the zeolite and to generate mesopores that are favorable to diffusion of the reactants and of the products of the sugar dehydration reaction.

The organic compound is advantageously in solution, preferably in aqueous solution, during contacting with the zeolite. Said solution preferably does not contain alkali metal or alkaline earth metal hydroxides such as NaOH. The use of NaOH or other alkali or alkaline earth metal hydroxides neutralizes the acidity of the zeolite and necessitates additional steps of retro-exchange of Na⁺ (or alkali or alkaline earth metal salts) after the base treatment, these additional steps making the process costly.

In one particular embodiment, said solution may comprise a second organic compound of formula:

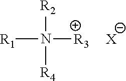

where R₁, R₂, R₃, and R₄ are independently selected from linear, branched or cyclic alkyl groups of 1 to 10 carbon atoms, and X⁻ is selected from the halide ions C⁻, Br⁻, and I⁻.

It is possible to employ a mixture of a plurality of cations N(R₁R₂R₃R₄)⁺, as well as a mixture of a plurality of anions X⁻.

In the process according to the invention it is important to make a distinction between the OH anion, which provides basicity and permits the formation of mesopores within the parent zeolite, from other anions such as halides.

Optionally, said solution may comprise an organic base, for example an amine such as dimethylamine or trimethylamine, as a second organic compound.

The contacting of the zeolite with the organic compound and optionally the second organic compound is preferably carried out in a container that permits stirring of the solution in order to ensure good material exchange.

The volume of the solution is defined in relation to the dry mass of zeolite used (i.e. parent zeolite). A volume of between 3 and 100 ml per gram of zeolite, preferably between 5 and 50 ml per gram of zeolite, is generally chosen.

The solution generally comprises a solvent which is water, optionally mixed with another protic and polar solvent.

The concentration of base in the solution corresponds to the sum of the molar amounts of N(R₁R₂R₃R₄)OH plus that of the second organic compound optionally added, per unit volume of solution. This concentration of base is advantageously between 0.1 and 2 mol/l, preferably between 0.1 and 0.3 mol/l.

The ratio between the concentration of N(R₁R₂R₃R₄)⁺ and the total concentration of base is between 1 and 0.1.

The final pH of the solution, i.e. at the end of the treatment, is preferably between 10 and 14.

The base treatment is advantageously performed at a temperature of between 2° and 90° C., preferably between 5° and 70° C.

The duration of the treatment is preferably between 15 min and 10 h, preferably between 1 and 4 h.

At the end of the treatment, the zeolite (also referred to as treated zeolite) is advantageously separated from the solution by filtration or by centrifugation.

Preferably, the zeolite resulting from the treatment (or from the final ion exchange) is separated and then dried at a temperature of between 8° and 150° C. (optionally by applying reduced pressure) and finally calcined at a temperature of between 50° and 650° C. in a stream of nitrogen or air. For reasons of safety (to avoid violent oxidation reactions of the organic compound) it is possible to raise the temperature in a stream of nitrogen before switching to a stream of air. The duration of the final constant temperature step is between 1 and 12 h.

The dehydration catalyst of the process according to the invention comprises the treated zeolite obtained after the base treatment and preferably separated, dried, and calcined. Said zeolite obtained after the base treatment, and preferably separated, dried and calcined, can optionally be mixed with a binder, to form said dehydration catalyst.

### Performance of the Dehydration Step

Said process includes a dehydration step. Said step of dehydrating the sugars is performed by contacting the hexose-containing feedstock with the dehydration catalyst in at least one solvent, preferably an organic solvent.

The process according to the invention is advantageously a process for producing 5-hydroxymethylfurfural from a hexose-containing feedstock.

The dehydration step of the process according to the invention, performed by contacting the hexose-containing feedstock with the dehydration catalyst in at least one solvent, may be carried out in a continuous or batchwise manner.

Said step of contacting the hexose-containing feedstock with the dehydration catalyst in at least one solvent is carried out at a temperature of between 3° and 160° C., preferably between 4° and 140° C., preferably between 5° and 120° C., more preferably 80 and 120° C., very preferably between 9° and 120° C., and at a pressure of between 0.0001 and 8.0 MPa, preferably between 0.001 and 5.0 MPa, and more preferably between 0.01 and 3.0 MPa.

The reaction medium comprises the hexose-containing feedstock, at least one solvent, preferably an organic solvent, and the dehydration catalyst.

The solvent is preferably a polar aprotic solvent. The polar aprotic solvent is advantageously selected from butan-2-one, acetone, acetic anhydride, N,N,N′,N′-tetramethylurea, benzonitrile, acetonitrile, methyl ethyl ketone, propionitrile, hexamethylphosphoramide, nitrobenzene, nitromethane, N,N-dimethylformamide, N,N-dimethylacetamide, sulfolane, N-methylpyrrolidone, dimethyl sulfoxide, propylene carbonate, and γ-valerolactone. Preferably, the polar aprotic solvent is selected from acetone, hexamethylphosphoramide, N,N-dimethylformamide, sulfolane, N-methylpyrrolidone, dimethyl sulfoxide, propylene carbonate, and γ-valerolactone.

Very preferably, the polar aprotic solvent is dimethyl sulfoxide (DMSO).

The advantage of using a polar aprotic solvent in the process is that the 5-HMF produced does not undergo rehydration; the rehydration of 5-HMF in a solvent such as water, for example, induces a loss of process selectivity for 5-HMF. The use of a polar aprotic solvent therefore makes it possible to obtain optimal process selectivity for 5-HMF.

An aprotic solvent is understood as meaning a molecule acting as solvent in which all the hydrogen atoms are borne on carbon atoms.

A polar solvent is understood as meaning a molecule acting as solvent, the dipole moment p of which, expressed in debye units, has a numerical value of greater than or equal to 2.00 at 25° C. according to databases known to those skilled in the art.

A polar aprotic solvent is thus understood as meaning a molecule acting as solvent in which all the hydrogen atoms are borne on carbon atoms and the dipole moment p of which, expressed in debye units, has a numerical value of greater than or equal to 2.00 at 25° C. according to databases known to those skilled in the art.

The feedstock is introduced into the reaction medium in a solvent/feedstock ratio by weight of between 0.1 and 200.0, preferably between 0.3 and 100.0, and more preferentially between 1.0 and 50.0.

Said dehydration step is advantageously fed by the hexose-containing feedstock in solid or liquid form. The feeding of the feedstock can be carried out according to several modes of introducing said feedstock.

In a first embodiment, the feedstock is introduced in solid form, optionally using a suitable device that makes it possible to control the feedstock flow rate. This device may be, but is not limited to, an endless screw or a pneumatic system for transporting solid particles. For a feedstock of oligosaccharide or polysaccharide type, this embodiment is preferred, but in a nonlimiting manner.

The introduction of a feedstock in solid form corresponding to sucrose, kestose or inulin, from which fructose is released by hydrolysis, is one possibility. Said introduction can be carried out one or more times in a sequential manner or else continuously.

In a second embodiment, the feedstock is introduced in liquid form in solution in a solvent, referred to as additional solvent, using a pump that permits control of the rate of introduction of the solution containing the feedstock. The additional solvent permits the dissolution of the sugars (or saccharides) in the feedstock.

The additional solvent may be selected from aprotic or protic polar solvents. Said additional solvent is preferably selected from butan-2-one, acetone, acetic anhydride, N,N,N′,N′-tetramethylurea, benzonitrile, acetonitrile, methyl ethyl ketone, propionitrile, hexamethylphosphoramide, nitrobenzene, nitromethane, N,N-dimethylformamide, N,N-dimethylacetamide, sulfolane, N-methylpyrrolidone, dimethyl sulfoxide, propylene carbonate, γ-valerolactone, water, methanol, ethanol, formic acid, and acetic acid.

The additional solvent selected from polar aprotic or protic solvents is preferably acetone, hexamethylphosphoramide, N,N-dimethylformamide, sulfolane, N-methylpyrrolidone, dimethyl sulfoxide, propylene carbonate, γ-valerolactone, water, methanol, and ethanol, preferably from N,N-dimethylformamide, sulfolane, N-methylpyrrolidone, dimethyl sulfoxide, water, and methanol, and very preferably the additional solvent is selected from water and dimethyl sulfoxide.

In this embodiment in which an additional solvent is present, the final 5-HMF concentration cannot exceed the limit of solubility of the sugar in the additional solvent, modified by the molar mass ratio of the fructose to the 5-HMF and modified by the dilution of the feedstock by the mass of polar aprotic solvent. The choice of additional solvent is therefore key in obtaining a high final 5-HMF concentration.

The content of the at least one additional solvent is between 0% and 50% by weight, preferably between 0% and 35% by weight, more preferably between 0% and 25% by weight, and very preferably between 0% and 20% by weight, relative to the total weight of solvent in the reaction medium.

This embodiment is particularly well suited to a feedstock of monosaccharide type, or even oligosaccharide type, which can be dissolved in the additional solvent in high concentrations.

Preference is given to a feedstock corresponding to a fructose syrup or a fructose and glucose syrup (of high-fructose corn syrup type) gradually introduced by means of a pump. Said introduction can be carried out one or more times in a sequential manner or else continuously, such that the instantaneous fructose concentration is maintained at less than or equal to 5.0% by weight.

The concentration of 5-HMF or fructose is understood as meaning the ratio of the weight of 5-HMF or fructose, respectively, to the weight of reaction medium.

In continuous operation of the process of the invention, the hourly mass flow rate (flow rate by weight of feedstock/weight of catalysts) is between 0.01 h⁻¹ and 5.0 h⁻¹ and preferably between 0.02 h⁻¹ and 2.0 h⁻¹.

Whichever embodiment is employed for the dehydration step of the process, the water generated and therefore present in the reaction mixture is preferably removed by any method known to those skilled in the art, preferably continuously, in order to maintain a water content of less than 30.0% by weight relative to the total weight of solvent, preferably less than 20.0% by weight, more preferably less than 15.0% by weight, and very preferably less than 10.0% by weight.

Advantageously, the operation of the 5-HMF production process, particularly through control of the instantaneous concentration, and in conjunction with the use of a zeolite modified according to the catalyst preparation step of the invention, makes it possible to obtain good conversion of the fructose involved, as well as excellent selectivity in favor of 5-HMF, and to improve 5-HMF productivity.

Thus, the selectivity, yield, and productivity obtained through the operation of the process of the invention make it possible, for example, to achieve final concentrations by weight of 5-HMF of greater than 3.5% by weight. The process according to the invention advantageously makes it possible to achieve final concentrations by weight of 5-HMF of greater than 5.0% by weight, preferably of greater than 10% by weight, and very preferably of greater than 15% by weight, relative to the weight of the reaction medium.

Products Obtained and their Method of Analysis

The product selectively obtained by the conversion process according to the invention is 5-hydroxymethylfurfural (5-HMF). At the end of the reaction performed in the process according to the invention, the reaction medium is analyzed by high-performance liquid chromatography (HPLC) to quantify the compounds present in the reaction medium, such as fructose and 5-HMF, but also undesired products such as levulinic acid, formic acid, and any sugar-containing co-products. The feedstock conversion and the fructose and 5-HMF content are then calculated.

The humins are quantified by difference in carbon balance with the carbon initially introduced.

### EXAMPLES

In the examples that follow, the fructose used as feedstock is a fructose that is commercially available and used without further purification.

The dimethyl sulfoxide, denoted DMSO in the examples, used as polar aprotic solvent, is commercially available and is used without further purification.

The concentrations by weight of the constituents of the reaction mixtures are determined by high-performance liquid chromatography (HPLC). Aliquots of the reaction mixture, i.e. samples, are withdrawn at regular intervals in order to evaluate the composition by HPLC.

HMF yields falling short of 100% are associated with only partial conversion of fructose or to the formation of undesired products (humins, polymeric species, etc.).

The fructose conversion (Conv_{FRU}) is defined as the ratio of the molar concentration of converted fructose to the molar concentration of fructoside units present in the initial feedstock, expressed as %.

The 5-HMF yield (Yield_{HMF}) is defined as the ratio of the molar concentration of 5-HMF measured in the samples to the molar concentration of fructoside units present only in the initial feedstock, expressed as %.

### Example 1: Preparation of the Dehydration Catalysts

The various samples are prepared by the same procedure, with only the parent zeolite and the nature of the tetraalkylammonium hydroxide changing.

The parent zeolite CBV720 used has an Si/Al molar ratio of 15.7 and a lattice parameter of 24.293 Å.

The parent zeolite CBV760 used has an Si/Al molar ratio of 27.2 and a lattice parameter of 24.259 Å.

300 ml of a 0.2 M aqueous tetraalkylammonium hydroxide solution is prepared with distilled water. When the solution reaches 65° C., 10 g of parent zeolite is introduced and the mixture is stirred at 400 rpm for 2 h at 65° C. The solution is quenched in ice-cold water, filtered, and washed 3 to 4 times with 300 ml portions of distilled water and centrifuged at 8000-10 000 rpm for 3 min. The solid recovered is dried in an oven at 120° C. overnight. The dried solid is ground and calcined at 550° C. for 4 h (1° C. min⁻¹) after being allowed to stand for 2 hours at 200° C. (1.5° C. min⁻¹).

A comparison solid is prepared according to the prior art (BC) with CBV760 as parent zeolite and treatment with NaOH and tetrapropylammonium bromide.

The parent zeolite and organic compound used for each sample preparation are summarized in the table below:

TABLE 1

| Solid | Parent zeolite | Organic compound (tetraalkylammonium) |
|---|---|---|
| A1 | CBV720 | Tetramethylammonium hydroxide |
| A2 | CBV720 | Tetraethylammonium hydroxide |
| A3 | CBV720 | Tetrapropylammonium hydroxide |
| A4 | CBV720 | Tetrabutylammonium hydroxide |
| B1 | CBV760 | Tetramethylammonium hydroxide |
| B2 | CBV760 | Tetraethylammonium hydroxide |
| B3 | CBV760 | Tetrapropylammonium hydroxide |
| BC | CBV760 | Tetrapropylammonium bromide |
| B4 | CBV760 | Tetrabutylammonium hydroxide |

The characteristics of the different parent zeolites and calculated samples (specific surface area, micropore volume, mesopore volume, crystallinity, and Brønsted acidity) are presented in the table below:

TABLE 2

| | | | | | | Brønsted | |
|---|---|---|---|---|---|---|---|
| | S_{BET} | V_{micro} | Vₘₑₛₒ ^{b} | Vₜₒₜₐₗ | Relative | acidity | Lewis |
| Solid | (m² g⁻¹) | (cm³ g⁻¹) | (cm³ g⁻¹) | (cm³ g⁻¹) | crystallinity | (μmol/g) | acidity |
| A CBV720 | 885 | 0.251 | 0.277 | 0.528 | 100% | 196 | 141 |
| A1 | 800 | 0.164 | 0.667 | 0.831 | 67% | 194 | 206 |
| A2 | 834 | 0.228 | 0.453 | 0.681 | 94% | 198 | 104 |
| A3 | 903 | 0.261 | 0.364 | 0.625 | 107% | 230 | 83 |
| A4 | 904 | 0.268 | 0.311 | 0.579 | 107% | 196 | 59 |
| B CBV760 | 859 | 0.251 | 0.322 | 0.573 | 100% | 125 | 41 |
| B1 | 895 | 0.224 | 0.713 | 0.936 | 0% | 78 | 119 |
| B2 | 882 | 0.249 | 0.4 | 0.650 | 58% | 129 | 108 |
| B3 | 946 | 0.235 | 0.781 | 1.016 | 92% | 174 | 52 |
| BC | 1219 | 0.289 | 0.991 | 1.280 | 68% | 76 | 38 |
| B4 | 882 | 0.249 | 0.400 | 0.650 | 100% | 153 | 56 |

N.D = Not defined

The BET specific surface area (SBET in m²/g) is determined by nitrogen adsorption in accordance with the ASTM 0-3663-78 standard, established on the basis of the Brunauer-Emmett-Teller method described in the journal “Journal of the American Chemical Society”, 1938, 60, 309.

The micropore volume is determined by the t-plot method using the Harkins-Jura method, with linear regression of the t-plot in the range between t=0.35 and 0.5 nm. The total pore volume corresponds to the volume adsorbed at saturation of the pores (N₂ relative pressure of 0.99). The mesopore volume is the difference between the total pore volume and the micropore volume.

The relative crystallinity of the zeolite is measured by X-ray diffraction. To evaluate crystallinity, the diffraction lines are integrated between 20 angles of 10 and 700. The ratio between the baseline-corrected integral and the baseline-uncorrected integral is calculated. This ratio is then normalized by the ratio of a reference zeolite (the parent zeolite before treatment).

A Brønsted acid is understood as meaning a compound capable of releasing a proton H⁺ in the reaction medium. A Lewis acid is understood as meaning a compound capable of accepting an electron pair.

The concentration of the Brønsted acid sites and Lewis acid sites in the zeolite is measured by pyridine adsorption. The zeolite is first dehydrated under reduced pressure at 450° C. and then exposed to pyridine vapor at 150° C. Excess pyridine is removed under reduced pressure. The concentration of Brønsted acid sites is determined by transmission IR spectroscopy, by integrating the band at 1540 cm⁻¹; the intensity is converted into mol/g by applying an extinction coefficient of 1.65 cm/pmol. The concentration of Lewis acid sites is determined by integrating the band at 1450 cm⁻¹, applying an extinction coefficient of 1.57 μmol/g.

Table 3 shows that the tetraalkylammonium hydroxide treatments result in good preservation of the zeolite micropore volume, especially with tetrapropylammonium and tetrabutylammonium hydroxides (A3, A4, B3, and B4). The mesopore volume is substantially increased in all cases.

Crystallinity is likewise very well preserved by the treatment, with the exception of B1. The acidity is close to or greater than that of the parent zeolite, with the exception of B1.

Treatment without NaOH and with tetraalkylammonium hydroxides generates zeolites having increased acidity compared to the prior art (BC).

### Example 2: Use of the Solids from Example 1 for Converting Fructose into 5-HMF

A 100 mL three-necked flask equipped with a condenser is charged with 2 g of crystalline fructose and 20 g of DMSO. The mixture is brought to a temperature of 90° C. with stirring. Once this temperature has been reached, 1.3 g of catalyst (i.e. solid prepared according to example 1) is added, which marks the commencement of the dehydration reaction. The mixture is then maintained at temperature with stirring. Samples are taken at various reaction times and analyzed by HPLC to quantify the fructose and 5-HMF concentrations, and to determine the HMF yield and conversion of fructose.

The results (fructose conversion and HMF yield after reaction times of 2 h and 6 h) are collated in the table below:

TABLE 3

| | CONV_{FRU} | Yield_{HMF} | CONV_{FRU} | Yield_{HMF} |
|---|---|---|---|---|
| Solid | at 2 h | at 2 h | at 6 h | at 6 h |
| A CBV720 | 40% | 8% | 51% | 21% |
| A1 | 42% | 4% | 51% | 17% |
| A2 | 43% | 8% | 54% | 25% |
| A3 | 54% | 23% | 74% | 46% |
| A4 | 53% | 23% | 74% | 47% |
| B CBV760 | 49% | 19% | 65% | 43% |
| B1 | 47% | 8% | 58% | 26% |
| B2 | 47% | 13% | 66% | 33% |
| B3 | 68% | 37% | 88% | 59% |
| B4 | 58% | 29% | 80% | 50% |

These results demonstrate that zeolites prepared with tetraalkylammonium hydroxides convert fructose. The reaction kinetics are particularly accelerated (fructose conversions and HMF yields higher after reaction times of 2 h and 6 h compared to the parent zeolites) for solids prepared with tetrapropylammonium hydroxide and tetrabutylammonium hydroxide (A3, A4, B3, and B4).

1. A process for dehydrating a hexose-containing feedstock, said process comprising:
preparing a dehydration catalyst by contacting a zeolite containing a series of channels in which the opening is greater than or equal to 10 oxygen atoms (10 MR) and having an Si/Al molar ratio of at least 10 with a solution comprising an organic compound of formula: 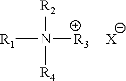
R₁, R₂, R₃, and R₄ being independently selected from linear, branched or cyclic alkyl groups of 1 to 10 carbon atoms, and X⁻ being the hydroxide ion OH⁻;
contacting said dehydration catalyst with said hexose-containing feedstock in at least one solvent, said contacting being carried out at a temperature of between 3° and 160° C. and at a pressure of between 0.0001 and 8.0 MPa.

2. The process as claimed in claim 1, wherein the hexose is fructose or a fructoside unit.

3. The process as claimed in claim 1, wherein the opening of the series of channels of the zeolite is equal to 12 oxygen atoms (12 MR).

4. The process as claimed in claim 3, wherein the zeolite is of FAU or EMT structural type.

5. The process as claimed in claim 4, wherein the zeolite is a USY zeolite.

6. The process as claimed in claim 1, wherein the zeolite has an Si/Al ratio of between 10 and 50.

7. The process as claimed in claim 1, wherein the organic compound comprises at least one group selected from methyl, ethyl, propyl, or butyl.

8. The process as claimed in claim 7, wherein the organic compound is selected from tetrapropylammonium hydroxide and tetrabutylammonium hydroxide.

9. The process as claimed in claim 1, wherein the solution comprising an organic compound does not contain NaOH.

10. The process as claimed in claim 1, wherein the solvent is a polar aprotic solvent.

11. The process as claimed in claim 1, wherein the zeolite has an Si/Al ratio of between 12 and 35.

12. The process as claimed in claim 1, wherein the hexose is fructose.

13. The process as claimed in claim 3, wherein the zeolite is of FAU structural type.
